(19) United States
(12) Patent Application Publication
(10) Pub. No.: US 2004/0077677 A1
(43) Pub. Date: Apr. 22, 2004

(54) Oral formulations

(75) Inventors: Muhammad Ashraf, Elmwood Park, NJ (US); Eric J. Benjamin, Pomona, NY (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(73) Assignee: Wyeth, Madison, NJ

(21) Appl. No.: 10/663,506

(22) Filed: Sep. 15, 2003

Related U.S. Application Data
(60) Provisional application No. 60/411,264, filed on Sep. 17, 2002.

(51) Int. Cl. A61K 31/4745

(52) U.S. Cl. 514/291

(57) ABSTRACT

This invention provides solid oral formulations of rapamycin 42-ester with 3-hydroxy-2-(hydroxymethyl)-2-methylpropionic acid (CCI-779).

### CROSS-REFERENCE TO RELATED APPLICATIONS

This is a non-provisional of U.S. Patent Application No. 60/411,264, filed Sep. 17, 2002, and claims the benefit of priority thereof.

### BACKGROUND OF THE INVENTION

This invention relates to oral solid formulations of rapamycin 42-ester with 3-hydroxy-2-(hydroxymethyl)-2-methylpropionic acid (CCI-779).

Rapamycin is a macrocyclic triene antibiotic produced by Streptomyces hygroscopicus, which was found to have antifungal activity, particularly against Candida albicans, both in vitro and in vivo [C. Vezina et al., J. Antibiot. 28, 721 (1975); S. N. Sehgal et al., J. Antibiot. 28, 727 (1975); H. A. Baker et al., J. Antibiot. 31, 539 (1978); U.S. Pat. No. 3,929,992; and U.S. Pat. No. 3,993,749]. Additionally, rapamycin alone (U.S. Pat. No. 4,885,171) or in combination with picibanil (U.S. Pat. No. 4,401,653) has been shown to have antitumor activity.

The immunosuppressive effects of rapamycin have been disclosed in FASEB 3, 3411 (1989). Cyclosporin A and FK-506, other macrocyclic molecules, also have been shown to be effective as immunosuppressive agents, therefore useful in preventing transplant rejection [U.S. Pat. No. 5,100,899]. R. Martel et al. [Can. J. Physiol. Pharmacol. 55, 48 (1977)] disclosed that rapamycin is effective in the experimental allergic encephalomyelitis model, a model for multiple sclerosis; in the adjuvant arthritis model, a model for rheumatoid arthritis; and effectively inhibited the formation of IgE-like antibodies.

Rapamycin is also useful in preventing or treating systemic lupus erythematosus [U.S. Pat. No. 5,078,999], pulmonary inflammation [U.S. Pat. No. 5,080,899], insulin dependent diabetes mellitus [U.S. Pat. No. 5,321,009], skin disorders, such as psoriasis [U.S. Pat. No. 5,286,730], bowel disorders [U.S. Pat. No. 5,286,731], smooth muscle cell proliferation and intimal thickening following vascular injury [U.S. Pat. Nos. 5,288,711 and 5,516,781], adult T-cell leukemia/lymphoma [European Patent Application 525,960 A1], ocular inflammation [U.S. Pat. No. 5,387,589], malignant carcinomas [U.S. Pat. No. 5,206,018], cardiac inflammatory disease [U.S. Pat. No. 5,496,832], and anemia [U.S. Pat. No. 5,561,138].

Rapamycin 42-ester with 3-hydroxy-2-(hydroxymethyl)-2-methylpropionic acid (CCI-779) is ester of rapamycin which has demonstrated significant inhibitory effects on ocular inflammation [U.S. Pat. No. 5,387,589], malignant carcinomas [U.S. Pat. No. 5,206,018], cardiac inflammatory disease [U.S. Pat. No. 5,496,832], and anemia [U.S. Pat. No. 5,561,138].

Rapamycin 42-ester with 3-hydroxy-2-(hydroxymethyl)-2-methylpropionic acid (CCI-779) is ester of rapamycin which has demonstrated significant inhibitory effects on tumor growth in both in vitro and in vivo models. The preparation and use of hydroxyesters of rapamycin, including CCI-779, are disclosed in U.S. Pat. No. 5,362,718.

CCI-779 exhibits cytostatic, as opposed to cytotoxic properties, and may delay the time to progression of tumors or time to tumor recurrence. CCI-779 is considered to have a mechanism of action that is similar to that of sirolimus. CCI-779 binds to and forms a complex with the cytoplasmic protein FKBP, which inhibits an enzyme, mTOR (mammalian target of rapamycin, also known as FKBP12-rapamycin associated protein [FRAP]). Inhibition of mTOR's kinase activity inhibits a variety of signal transduction pathways, including cytokine-stimulated cell proliferation, translation of mRNAs for several key proteins that regulate the G1 phase of the cell cycle, and L-2-induced transcription, leading to inhibition of progression of the cell cycle from G1 to S. The mechanism of action of CCI-779 that results in the G1 to S phase block is novel for an anticancer drug.

In vitro, CCI-779 has been shown to inhibit the growth of a number of histologically diverse tumor cells. Central nervous system (CNS) cancer, leukemia (T-cell), breast cancer, prostate cancer, and melanoma lines were among the most sensitive to CCI-779. The compound arrested cells in the G1 phase of the cell cycle.

In vivo studies in nude mice have demonstrated that CCI-779 has activity against human tumor xenografts of diverse histological types. Gliomas were particularly sensitive to CCI-779 and the compound was active in an orthotopic glioma model in nude mice. Growth factor (platelet-derived)-induced stimulation of a human glioblastoma cell line in vitro was markedly suppressed by CCI-779. The growth of several human pancreatic tumors in nude mice as well as one of two breast cancer lines studied in vivo also was inhibited by CCI-779.

One obstacle towards the formulation of CCI-779 is its poor aqueous solubility (less than 1 μg/ml), which makes its bioavailability low. In additional, CCI-779 exhibits aqueous instability via cleavage of a lactone bond, resulting in the formation of the ring opened seco-CCI-779. CCI-779 tablets prepared by direct compression of non-micronized CCI-779 with standard excipients and fillers, in the presence or absence of surfactants provided tablets which did not exhibit rapid and complete drug release, and thereby provided an unsuitable formulation for CCI-779.

### SUMMARY OF THE INVENTION

This invention avoids the aforementioned problems by employing a water-soluble polymer such as povidone (PVP) and employing a wet granulation process to provide a highly bioavailable non-micronized CCI-779 formulation that overcomes the dissolution and instability problem. The inhibition of degradation can also be assisted by the use of one or more antioxidants, and a pH modifying agent to maintain a pH of about 4 to about 6.

### DETAILED DESCRIPION OF THE INVENTION

Accordingly, this invention provides a solid formulation comprising a granulation prepared using a wet granulation process, said granulation comprising CCI-779, a water soluble polymer, a pH modifying agent, a surfactant, and an antioxidant. In one embodiment, the formulation contains from 0.1 to 30%, from 0.5 to 25%, from 1 to 20%, from 5 to 15%, or from 7 to 12% (wt/wt) CCI-779, from 0.5 to 50%, from 1 to 40%, from 5 to 35%, from 10 to 25%, or from 15 to 20% (wt/wt) water soluble polymer, from 0.5 to 10%, 1 to 8%, or 3 to 5% (wt/wt) surfactant, and from 0.001% to 1%, 0.01% to 1%, or 0.1% to 0.5% (wt/wt) antioxidant. However, other embodiments may contain more, or less, of these components.

The formulation may also contain suitable chelating agents, fillers, binders, surfactants, and the like to facilitate the granulation and tableting process. It is preferred that the wet granulation be performed with a hydroalcoholic solvent system comprising water and an alcohol, with ethanol being the preferred alcoholic component.

Typical water soluble polymers include, but are not limited to, polyvinylpyrrolidone (PVP), hydroxypropylmethylcellulose (HPMC), polyethylene glycol (PEG), and cyclodextrins. It is preferred that the water soluble polymer is PVP, and having a molecular weight of between 2.5 and 60 kilodaltons. Any given formulation of this invention may contain multiple ingredients of each class of component. For example, a formulation containing an antioxidant may contain one or more antioxidants as the antioxidant component.

Acceptable pH modifying agents include, but are not limited to citric acid, sodium citrate, dilute HCl, and other mild acids or bases capable of buffering a solution containing CCI-779 to a pH in the range of about 4 to about 6.

Acceptable antioxidants include, but are not limited to, citric acid, d,I-α-tocopherol, BHA, BHT, monothioglycerol, ascorbic acid, and propyl gallate. It is expected that the antioxidants of the formulations of this invention will be used in concentrations ranging from 0.001% to 3% wt/wt.

Chelating agents, and other materials capable of binding metal ions, such as ethylene diamine tetra acetic acid (EDTA) and its salts are capable of enhancing the stability of CCI-779.

Surfactants may include polysorbate 80, sodium lauryl sulfate, sodium dodecyl sulfate, salts of bile acids (taurocholate, glycocholate, cholate, deoxycholate, etc.) which may be combined with lecithin. Alternatively, ethoxylated vegetable oils, such as Cremophor EL, vitamin E tocopherol propylene glycol succinate (Vitamin E TGPS), polyoxyethylene-polyoxypropylene block copolymers, and poloxamers.

Binders, fillers, and disintegrants such as sucrose, lactose, microcrystalline cellulose, croscarmellose sodium, magnesium stearate, gum acacia, cholesterol, tragacanth, stearic acid, gelatin, casein, lecithin (phosphatides), carboxymethylcellulose calcium, carboxymethylcellulose sodium, methylcellulose, hydroxyethylcellulose, hydroxypropylcellulose, hydroxypropylmethycellulose phthalate, noncrystalline cellulose, cetostearyl alcohol, cetyl alcohol, cetyl esters wax, dextrates, dextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, and polyvinyl alcohol, and the like may also be incorporated into the formulation.

The formulation can be prepared by preparing an alcoholic solution comprising CCI-779 and an antioxidant, and an aqueous solution comprising a water soluble polymer, a surfactant, and a pH modifier, in sufficient quantity to adjust the pH of the aqueous solution to 4 to 6. Suitable alcohols include methanol, ethanol, isopropanol, and the like, where ethanol is the preferred alcohol. The solutions were mixed and added to a mixer containing intragranular excipients. Alternatively, the alcoholic and aqueous solutions can be added separately without mixing with each other. Such intragranular excipients comprise binders and fillers to promote dissolution enhancement. Typical intragranular excipients may include, but are not limited to, microcrystalline cellulose, lactose, and croscarmellose sodium. The solid intragranular excipients are granulated with the solutions in the mixer until a uniform granulation is achieved. The mixer can be a blender with intensifying bar, a low shear granulator or a high shear granulator. The granulation is dried in a fluid bed dryer at approximately 50° C., and milled using a suitable milling device, such as a Fitz mill. The wet granulation and drying can be done in a fluid bed granulator/dryer. The wet granulation can be dried using an tray drying oven. If desired, the dried granulation can be further blended with exteragranular fillers and binders, such as microcrystalline cellulose, croscarmellose sodium, and magnesium stearate in a blender, such as a V-blender, before compression into tablets.

Alternatively, some of the water-soluble polymer can be contained in the intragranular excipients, and the aqueous and alcoholic solutions added to the mixer containing the intragranular excipients stepwise. For example, the order of addition to the mixer may be one half of the aqueous solution, followed by the entire alcoholic solution, and then the remainder of the aqueous solution. Other sequences of addition are possible and permissible under this invention.

The following provide representative examples of the formulations of this invention. The preparation of CCI-779 is described in U.S. Pat. No. 5,362,718, which is hereby incorporated by reference. A regioselective preparation of CCI-779 is described in U.S. Pat. No. 6,277,983, which is hereby incorporated by reference. These examples are illustrative only, and do not limit the invention.

### EXAMPLES

Procedure A

The following procedure was used to prepare a tablet containing 2 mg CCI-779 containing the following components; quantities are adjusted to account for low potency:

| Ingredient | Percent Wt/Wt |
|---|---|
| CCI-779 | 1.77 |
| Butylated Hydroxyanisole | 0.10 |
| Butylated Hydroxytoluene | 0.05 |
| PVP, 17PF | 8.85 |
| Edetic Acid | 0.01 |
| Sodium Lauryl Sulfate | 3.0 |
| Sodium Citrate (anhydrous) | 0.75 |
| Citric Acid (anhydrous) | 0.25 |
| Microcrystalline Cellulose | 50.4 |
| Croscarmellose Sodium | 4.0 |
| Anhydrous Lactose | 30.32 |
| Magnesium Stearate | 0.5 |
| Dehydrated Alcohol (ethanol)* | |
| Purified Water* | |

*Used in processing, but does not appear in final product.

Microcrystalline cellulose, anhydrous lactose, and croscarmellose sodium were screened through a 20 mesh screen, transferred to a V-Blender with an intensifying bar and mixed. Sodium lauryl sulfate, edetic acid, sodium citrate, citric acid, and PVP were dissolved in a sufficient quantity of purified water to achieve a solution. Butylated hydroxyanisole, butylated hydroxytoluene, and CCI-779 were dissolved in a sufficient quantity of dehydrated alcohol to achieve a solution. The alcohol solution was added to the aqueous solution with stirring. The alcoholic solution container was washed with dehydrated alcohol, which was added to the hydroholic solution. The solution was stirred until a clear solution resulted. The hydroholic solution was added to the V-Blender and the ingredients granulated. The hydroholic solution container was washed with a 10% alcohol solution that was added to the granulation. The granulation was mixed until uniformity was achieved, followed by drying in a fluid bed dryer. The dried granulation was passed through a 30 mesh screen, and any oversized granulation milled through a Fitzmill. The milled granulation was transferred to a V-Blender. Additional microcrystalline cellulose, anhydrous lactose, and croscarmellose sodium were passed through a 20 mesh screen and added to the blender. The mixture was blended, magnesium stearate (screened through a 30 mesh screen) was added to the blender, and the mixture blended. The resulting mixture was compressed into tablets.

The following table shows a comparison of the dissolution in water of (a) pure CCI-779 in capsules, (b) a tablet of the dry blend of the same ingredients contained in the granulation, and (c) a tablet of the granulation prepared as described above. The results clearly demonstrate that the hydroholic granulation of this invention provided enhanced dissolution in water, and will thereby provide enhanced bioavailability.

| | Percent CCI-779 Dissolved | | |
|---|---|---|---|
| | | CCI-779 | CCI-779 |
| Time (min) | CCI-779 Capsules | Dry Blend Tablet | Wet Granulation |
| 10 | 4 | 31 | 96 |
| 20 | 9 | 42 | 104 |
| 30 | 14 | 50 | 104 |
| 45 | 21 | 56 | 104 |

Procedure B

The following procedure was used to prepare a tablet containing 25 mg CCI-779 containing the following components; quantities are adjusted to to account for low potency:

| Ingredient | Percent Wt/Wt |
|---|---|
| CCI-779 | 4.0 |
| Butylated Hydroxyanisole | 0.10 |
| Butylated Hydroxytoluene | 0.05 |
| PVP, 17PF | 21.0 |
| Edetic Acid | 0.01 |
| Sodium Lauryl Sulfate | 3.6 |
| Citric Acid (anhydrous) | 0.025 |
| Microcrystalline Cellulose | 44.5 |
| Croscarmellose Sodium | 4.0 |
| Anhydrous Lactose | 22.17 |
| Magnesium Stearate | 0.5 |
| Dehydrated Alcohol (ethanol)* | |
| Purified Water* | |

*Used in processing, but does not appear in final product.

Microcrystalline cellulose, anhydrous lactose, approximately one half of the PVP, and croscarmellose sodium were screened through a 20 mesh screen, transferred to a V-Blender with an intesifying bar and mixed. Sodium lauryl sulfate, edetic acid, citric acid, and the remaining PVP were dissolved in a sufficient quantity of purified water to achieve a solution. The pH of the solution was measured, and if higher than 4.5, it was lowered with 0.1 N HCl until a pH of 4.5 was achieved. Butylated hydroxyanisole, butylated hydroxytoluene, and CCI-779 were dissolved in a sufficient quantity of dehydrated alcohol to achieve a solution. About one half of the aqueous solution was added to the blender, and the granulation mixed for about 4 minutes. The alcoholic solution was added to the blender and the granulation mixed for about 4 minutes. The remaining aqueous solution was added to the blender and the granulation mixed for about 4 minutes. Additional water was added, if needed to make a uniform granulation. The granulation was dried in a fluid bed dryer at a temperature of about 50° C. The dried granulation was passed through a 30 mesh screen, and any oversized granulation milled through a Fitzmill. The milled granulation was transferred to a V-Blender. Additional microcrystalline cellulose, anhydrous lactose, and croscarmellose sodium were passed through a 20 mesh screen and added to the blender. The mixture blended and magnesium stearate (screened through a 30 mesh screen) was added to the blender, and the mixture blended. The resulting mixture was compressed into tablets.

The documents cited throughout this specification are hereby incorporated by reference. Minor variations and modifications to the methods and materials set forth in the foregoing detailed description and illustrative examples will be readily apparent to those of skill in the art and are encompassed within the scope of the invention.

What is claimed is:

1. A pharmaceutical composition for oral administration comprising a granulation, said granulation comprising CCI-779, a water soluble polymer, a surfactant, an antioxidant, and a pH modifying agent.

2. The composition of claim 1, wherein the water soluble polymer is PVP, hydroxypropylmethylcellulose, polyethylene glycol, or cyclodextrin or mixtures thereof.

3. The composition of claim 2, wherein the water soluble polymer is PVP.

4. The composition of claim 3, wherein the surfactant is polysorbate 80, sodium lauryl sulfate, sodium dodecyl sulfate, a salt of a bile acid, an ethoxylated vegetable oil, a polyoxyethylene-polyoxypropylene block copolymer, or a poloxamer.

5. The composition of claim 4, wherein the surfactant is sodium lauryl sulfate or sodium dodecyl sulfate.

6. The pharmaceutical composition of claim 5, wherein the pH modifying agent is sodium citrate, citric acid, or dilute hydrochloric acid.

7. A process for preparing a CCI-779 oral composition, which comprises
(a) dissolving CCI-779 and an antioxidant in an alcohol to form an alcoholic solution;
(b) dissolving PVP, a pH modifying agent, and a surfactant in water to form an aqueous solution;
(c) mixing the alcoholic solution and the aqueous solution to form a hydroholic solution.
(d) adding the hydroholic solution to a mixer containing one or more intragranular excipients;
(e) granulating the mixture; and
(f) drying the resulting granulation.

8. A process for preparing a CCI-779 oral composition, which comprises
(a) dissolving CCI-779 and an antioxidant in an alcohol to form an alcoholic solution;
(b) dissolving PVP, a pH modifying agent, and a surfactant in water to form an aqueous solution;
(c) adding the aqueous and alcoholic solutions stepwise, and in one or more portions each, to a mixer containing one or more intragranular excipients;
(e) granulating the mixture; and
(f) drying the resulting granulation.

9. A CCI-779 oral composition prepared by wet granulation.

10. A CCI-779 oral composition prepared the process comprising
(a) dissolving CCI-779 and an antioxidant in an alcohol;
(b) dissolving PVP, a pH modifying agent, and a surfactant in water;
(c) combining the aqueous and alcoholic solutions to provide a hydroholic solution;
(d) adding the hydroalcoholic solution to a mixer containing one or more intragranular excipients;
(e) granulating the mixture; and
(f) drying the resulting granulation.

11. The composition of claim 10, wherein the pH modifying agent is selected from the group consisting of citric acid, sodium citrate, hydrochloric acid and mixtures thereof.

12. The composition of claim 11, wherein the alcohol is ethanol.

13. The composition of claim 12, wherein the antioxidant is butylated hydroxyanisole and butylated hydroxytoluene.

14. The composition of claim 13, wherein the surfactant is sodium lauryl sulfate.

15. A CCI-779 oral formulation prepared by the process comprising
(a) dissolving CCI-779 and an antioxidant in an alcohol;
(b) dissolving PVP, a pH modifying agent, and a surfactant in water;
(c) adding the aqueous and alcoholic solutions stepwise, and in one or more portions each, to a mixer containing one or more intragranular excipients;
(e) granulating the mixture; and
(f) drying the resulting granulation.

16. The composition of claim 15, wherein the pH modifying agent is selected from the group consisting of citric acid, sodium citrate, hydrochloric acid and mixtures thereof.

17. The composition of claim 16, wherein the alcohol is ethanol.

18. The composition of claim 17, wherein the antioxidant is butylated hydroxyanisole and butylated hydroxytoluene.

19. The composition of claim 18, wherein the surfactant is sodium lauryl sulfate.
